(19) United States
(12) Patent Application Publication
(10) Pub. No.: US 2025/0110693 A1
(43) Pub. Date: Apr. 3, 2025

(54) MEASURING APPARATUS, MEASURING METHOD, AND PROGRAM

(71) Applicant: JVCKENWOOD Corporation, Yokohama-shi (JP)

(72) Inventors: Kuniaki TAKACHI, Yokohama-shi (JP); Hisako MURATA, Yokohama-shi (JP); Makoto ORYOJI, Yokohama-shi (JP); Yoichi HARUKAWA, Yokohama-shi (JP); Toru KAIWA, Yokohama-shi (JP); Takayuki UCHIDA, Yokohama-shi (JP)

(21) Appl. No.: 18/980,748

(22) Filed: Dec. 13, 2024

Related U.S. Application Data
(63) Continuation of application No. PCT/JP2023/023541, filed on Jun. 26, 2023.

(30) Foreign Application Priority Data
Jul. 13, 2022 (JP) 2022-112550

(51) Int. Cl. G06F 3/16

(52) U.S. Cl. CPC G06F 3/167; G06F 3/162; G06F 3/165

(57) ABSTRACT

A measuring apparatus according to the present disclosure measures the hearing ability of a user by performing a binary tree search based on responses of the user to measurement sounds. The measuring apparatus includes a first search processing unit configured to select, as a selected group, one of a plurality of groups obtained by dividing a measurement range of the hearing ability into the plurality of groups; and a second search processing unit configured to repeat the binary tree search in the selected group until the binary tree search converges, and thereby determine the hearing ability.

### CROSS REFERENCE TO RELATED APPLICATION

This application is based upon and claims the benefit of priority from International Patent Application No. PCT/JP2023/023541 filed on Jun. 26, 2023, which is based on Japanese patent application No. 2022-112550, filed on Jul. 13, 2022, the disclosure of which is incorporated herein in its entirety by reference.

### BACKGROUND

The present disclosure relates to a measuring apparatus, a measuring method, and a program.

A technology in which a plurality of measurement sounds having different volumes are output from an earphone or the like, and the hearing ability of a user, who is the subject of the measurement, is measured based on responses of the user to the measurement sounds has been known.

As a related technology, for example, Patent Literature 1 discloses an acoustic signal processing apparatus including a hearing-ability measurement unit that outputs measurement sounds and measures the hearing characteristics of a user. In this apparatus, the hearing-ability measurement unit presents sounds to the user at various levels for each octave. The hearing-ability measurement unit can determine the level of the presented sound by using, for example, a binary tree search method. The user presses an OK button provided in an operation unit only when he/she could hear the presented sound. The hearing-ability measurement unit determines, among the presented levels, the minimum level at which the OK button is pressed as the minimum audible level of the user. In this way, the acoustic signal processing apparatus disclosed in Patent Literature 1 can measure the hearing characteristics of the user based on the response from the user.
Patent Literature 1: Japanese Unexamined Patent Application Publication No. 2012-213114

### SUMMARY

Assume that the hearing ability of a user is measured by using the binary tree search method as described in the aforementioned technology. While the processing can be performed at a high speed by using the binary tree search method, there is a problem that a correct measurement result cannot be obtained when the user mistakenly responds.

For example, assume that in the example shown in Patent Literature 1, the user presses the OK button by mistake even though he/she could not hear the measurement sound. In this case, the acoustic signal processing apparatus cannot correctly perform the binary tree search, so that an appropriate measurement result may not be obtained. In particular, when the user mistakenly responds to the first measurement sound among a plurality of measurement sounds presented to the user, there is a possibility that the measurement result will be widely deviated from the correct one.

A measuring apparatus according to an embodiment is a measuring apparatus configured to measure a hearing ability of a user by performing a binary tree search based on responses of the user to measurement sounds, including:
a first search processing unit configured to select, as a selected group, one of a plurality of groups obtained by dividing a measurement range of the hearing ability into the plurality of groups; and
a second search processing unit configured to repeat the binary tree search in the selected group until the binary tree search converges, and thereby determine the hearing ability.

A measurement method according to an embodiment is a method for measuring a hearing ability of a user by performing a binary tree search based on responses of the user to measurement sounds, including:
a first search processing step of selecting, as a selected group, one of a plurality of groups obtained by dividing a measurement range of the hearing ability into the plurality of groups; and
a second search processing step of repeating the binary tree search in the selected group until the binary tree search converges, and thereby determining the hearing ability.

A program according to an embodiment is a program for causing a computer of a measuring apparatus configured to measure a hearing ability of a user by performing a binary tree search based on responses of the user to measurement sounds to perform a measurement method, the measurement method including:
a first search processing step of selecting, as a selected group, one of a plurality of groups obtained by dividing a measurement range of the hearing ability into the plurality of groups; and
a second search processing step of repeating the binary tree search in the selected group until the binary tree search converges, and thereby determining the hearing ability.

### BRIEF DESCRIPTION OF THE DRAWINGS

The above and other aspects, advantages and features will be more apparent from the following description of certain embodiments taken in conjunction with the accompanying drawings, in which:

FIG. 1 is a block diagram showing a configuration of a measuring apparatus according to an embodiment;

FIG. 2 shows an example of a display window output to an input/output unit according to an embodiment;

FIG. 3 shows a first binary tree according to an embodiment;

FIG. 4 shows a second binary tree according to an embodiment;

FIG. 5 is a flowchart showing a measurement process performed by a measuring apparatus according to an embodiment; and

FIG. 6 shows a binary tree used in a related art.

### DETAILED DESCRIPTION

Embodiments according to the present disclosure will be described hereinafter in detail with reference to the drawings. The same or corresponding elements are assigned the same reference numerals (or symbols) throughout the drawings. For clarifying the explanation, redundant descriptions are omitted as appropriate.

### Description of Problem to be Solved

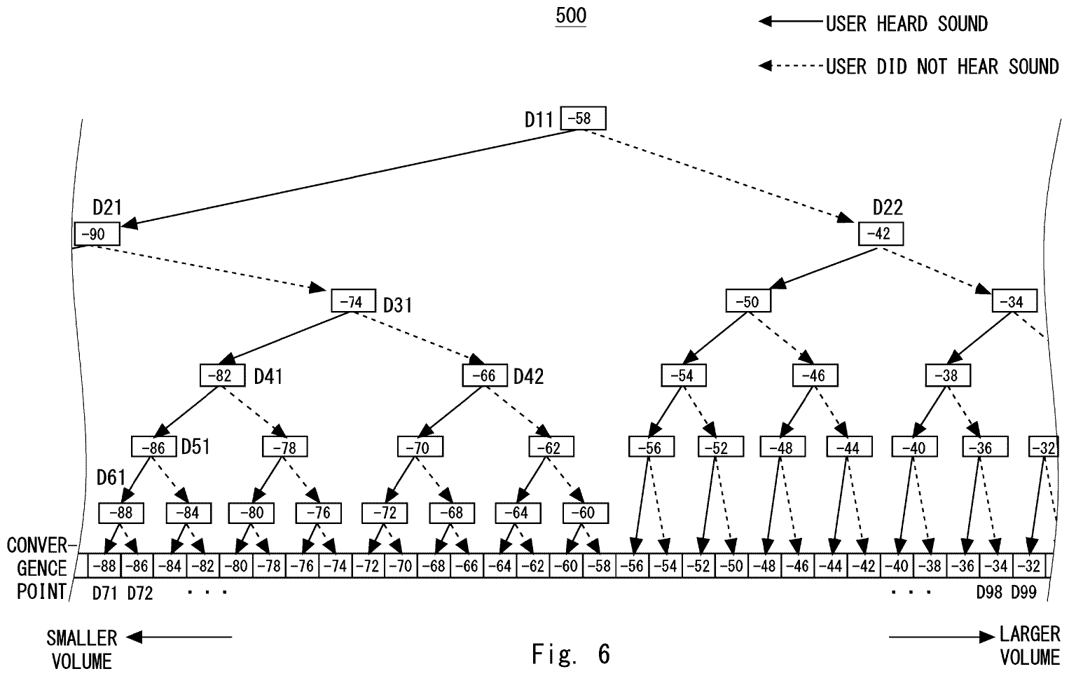

Firstly, a problem to be solved by the present disclosure will be described in a concrete manner with reference to FIG. 6. FIG. 6 shows a binary tree 500 used in a related art. Note that it is assumed that a measuring apparatus 10a (not shown) measures the hearing ability of a user. The binary tree 500 is stored in a storage device or the like provided in the measuring apparatus 10a, and the measuring apparatus 10a performs a search in the binary tree 500. In this way, the measuring apparatus 10a performs a measurement process for measuring the hearing ability of the user.

Similarly to a measuring apparatus 10 according to an embodiment of the present disclosure (which will be described later), the measuring apparatus 10a includes a sound output unit 13 and an input/output unit 14. The sound output unit 13 is an output device for outputting measurement sounds. The sound output unit 13 is, for example, an earphone. Further, the input/output unit 14 is an input/output device for receiving responses to the measurement sounds from the user. The response to a measurement sound may be information indicating whether or not the user could hear the measurement sound. The input/output unit 14 is, for example, a display device equipped with a touch panel that enables a user to perform an input operation by touching the touch panel with his/her finger(s) or the like. Note that in the following description, a measurement sound may also be simply referred to as a “sound”.

As shown in FIG. 6, the binary tree 500 has nodes D11, D21, D22, . . . , and D99. The binary tree 500 has a binary tree structure used for measuring the hearing ability of a user. Note that although the binary tree 500 extends beyond the left and right sides of the range shown in the drawing, they are omitted in the drawing.

In FIG. 6, each node is represented by a rectangular frame. Further, for each node, the volume level of a measurement sound associated with that node is shown inside the frame. The unit of the volume level is dB. When the measuring apparatus 10a reaches a given node, it outputs a measurement sound having a volume level corresponding to this node.

For example, the measuring apparatus 10a starts a measurement process by using the highest-level node D11 as a starting point, and a volume level of −58 dB is associated with this node D11. Therefore, the measuring apparatus 10a first outputs a measurement sound having the volume level of −58 dB from the sound output unit 13.

As shown in FIG. 6, the binary tree 500 is configured so that a node branches according to whether the user could hear the measurement sound or could not hear the measurement sound. In FIG. 6, cases where the user could hear the measurement sound are indicated by solid-line arrows, whereas cases where the user could not hear the measurement sound are indicated by broken-line arrows.

The measuring apparatus 10a outputs, at each node, a measurement sound corresponding to that node, and repeats a binary tree search according to the response of the user to the measurement sound. The measuring apparatus 10a ends the search when it reaches the node at the convergence point. The measuring apparatus 10a determines the hearing ability of the user based on the volume level corresponding to the node at the convergence point. In this way, the measuring apparatus 10a can measure the hearing ability of the user.

Therefore, the measuring apparatus 10a can measure the hearing ability of the user by using the range in which the convergence points are set as a measurement range. The measurement range can correspond to, for example, a range of volumes with which the measuring apparatus 10a can output a sound from the sound output unit 13 (earphone). The measurement range can be set by using the maximum volume and the minimum volume among the volumes with which the measuring apparatus 10a can output a sound from the sound output unit 13. Note that since the left and right sides of the binary tree 500 are partially omitted in FIG. 6, the maximum volume and the minimum volume with which the measuring apparatus 10a can output a sound from the sound output unit 13 are not shown in the drawing. Assume that, for example, the maximum volume is −26 dB and the minimum volume is −120 dB in the binary tree 500. In this case, the measurement range is from −120 dB to −26 dB. Note that the measurement range may be set to a range different from the above-described range of volumes with which the measuring apparatus 10a can output a sound from the sound output unit 13. For example, the measurement range may be set to a range smaller than the range of volumes with which the measuring apparatus 10a can output a sound from the sound output unit 13.

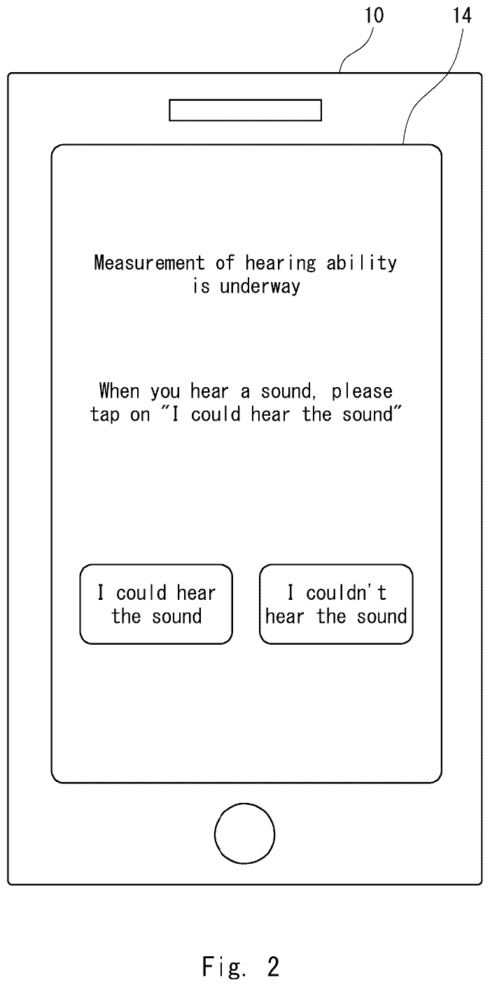

Processes that the measuring apparatus 10a performs to measure the hearing ability of a user by using the binary tree 500 will be described. The measuring apparatus 10a first outputs a measurement sound having a volume level of −58 dB corresponding to the highest-level node D11 of the binary tree 500 from the sound output unit 13. The user inputs, to the measuring apparatus 10a through the input/output unit 14, whether or not he/she could hear the measurement sound output from the sound output unit 13. It is assumed that a display window shown in FIG. 2 is displayed on the input/output unit 14 (which will be described later).

When the user determines that he/she could hear the measurement sound, he/she operates the input/output unit 14 and presses an “I could hear the sound” button. Further, when the user determines that he/she could not hear the measurement sound, he/she operates the input/output unit 14 and presses an “I couldn't hear the sound” button. Alternatively, when the user has not operated the input/output unit 14 for a predetermined time (e.g., five seconds), the measuring apparatus 10a determines that the user could not hear the measurement sound. In this way, the measuring apparatus 10a receives a response from the user.

Here, it is assumed that the user has responded with information indicating that he/she could hear the measurement sound of the node D11. The measuring apparatus 10a proceeds from the node D11 of the binary tree 500 to a node D21 thereof, and outputs a measurement sound having a volume level of −90 dB. It is assumed that the user has responded to this measurement sound with information indicating that he/she could not hear the measurement sound. In response to this response of the user, the measuring apparatus 10a proceeds to a node D31 and outputs a measurement sound having a volume level of −74 dB.

The measuring apparatus 10a repeatedly performs the above-described processes and thereby proceeds with the search in the binary tree 500. For example, assume that in the subsequent process, the measuring apparatus 10a proceeds with the binary tree search in the order of nodes D41, D51, D61 and D71. The measuring apparatus 10a obtains a volume level of −88 dB corresponding to the node D71, which is the convergence point. The measuring apparatus 10a determines the hearing ability of the user based on this volume level of −88 dB.

The measuring apparatus 10a according to the related art measures the hearing ability of the user by the above-described measurement process. However, in the measurement using a binary tree such as the binary tree 500, there is a possibility that a correct measurement result is not obtained when the user could not correctly input a response to a measurement sound to the measuring apparatus 10a.

For example, there is a case where the user mistakenly presses the “I could hear the sound” button even though he/she could not hear the measurement sound. Further, there is a case where the user mistakenly presses the “I couldn't hear the sound” button even though he/she could hear the measurement sound. Further, there is a case where the user thinks that he/she has correctly pressed the “I could hear the sound” button or the “I couldn't hear the sound” button, but in reality, he/she has not correctly pressed the button. Further, in addition to such mistakes in regard to the pressing of the button, there is a conceivable case where the user mistakenly recognizes that he/she has heard the measurement sound due to an ambient sound or the like even though, in reality, he/she did not hear the measurement sound.

When such a mistake in regard to the pressing of the button or a mistake in which the user has incorrectly recognized other sounds as the measurement sound, the user responds to the measuring apparatus 10a with incorrect information. In such a case, the measuring apparatus 10a cannot accurately measure the hearing ability of the user. In particular, when such an incorrect response occurs at an early stage of the binary tree search, there is a possibility that the measurement result will be widely deviated from the correct one.

For example, assume that in the above-described example, the user has pressed the “I couldn't hear the sound” button even though he/she heard the measurement sound at the node D11. In this case, although the measuring apparatus 10a should proceed to the node D21, it proceeds to the node D22 according to the incorrect response of the user and performs processes described below. The range of convergence points of the nodes lower than the node D21, i.e., nodes branched from the node D21, differs from that of the node D22. Therefore, the measuring apparatus 10a cannot accurately measure the hearing ability of the user. The measuring apparatus 10 according to the present disclosure is designed to cope with such a problem.

### (Configuration of Measurement Apparatus 10)

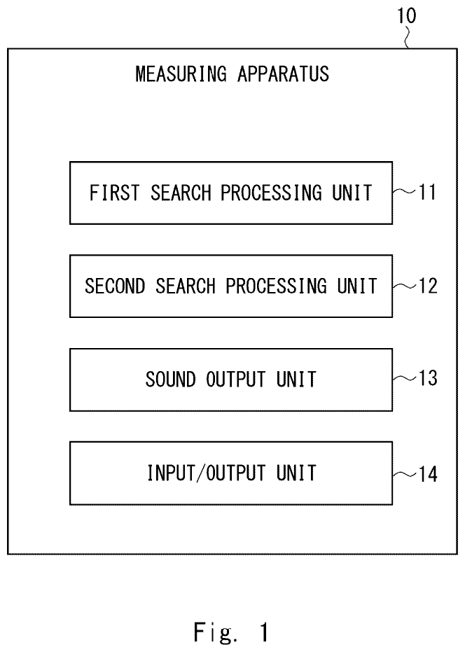

Next, the measuring apparatus 10 according to this embodiment will be described with reference to FIG. 1. FIG. 1 is a block diagram showing the configuration of the measuring apparatus 10. As shown in FIG. 1, the measuring apparatus 10 includes a first search processing unit 11, a second search processing unit 12, a sound output unit 13, and an input/output unit 14. The basic flow of the binary tree search in the processes according to this embodiment is substantially the same as the above-described flow of the binary tree search. Therefore, redundant descriptions may be omitted as appropriate in the following description.

The measuring apparatus 10 is an apparatus that measures the hearing ability of a user by performing a binary tree search based on responses of the user to measurement sounds. Specifically, the measuring apparatus 10 outputs a plurality of measurement sounds having different volume levels according to a predetermined binary tree. The measuring apparatus 10 acquires a response of the user to each measurement sound, and performs the binary tree search based on the content, i.e., information, indicated by the response. The content of the response can be expressed as “the user could hear the sound” or “the user could not hear the sound” as being expressed in the example described above.

The measuring apparatus 10 can be used in various apparatuses for measuring the hearing ability of a user. The measuring apparatus 10 can be used, for example, in an audio receiving apparatus that receives sounds from a television set, a DVD player, or the like, and adjusts the volume of sounds output therefrom according to the hearing ability of the user. As the measuring apparatus 10 measures the hearing ability of the user, it can adjust the volume based on the result of this measurement. The use of the measuring apparatus 10 is not limited to the above-described example, and the measuring apparatus 10 may be used in a telephone apparatus or the like. Further, the measuring apparatus 10 may be used in a hearing-ability test or the like for simply measuring a hearing ability.

The measuring apparatus 10 may or may not be implemented by using a dedicated apparatus. The measuring apparatus 10 may be formed by using, for example, a smartphone, a mobile phone terminal, a tablet terminal, a PC (Personal Computer), or the like. In this embodiment, an example in which the measuring apparatus 10 is implemented in an information terminal such as a smartphone will be described. For the measuring apparatus 10, the aforementioned information terminal may be implemented as the measuring apparatus 10 by installing a predetermined application in the information terminal.

The measuring apparatus 10 includes a processor, a memory, and a storage device as its configuration (not shown). In the storage device, a computer program in which processes according to this embodiment are implemented is stored. The processor can load the computer program from the storage device onto the memory, and execute the loaded computer program. In this way, the processor implements the functions of the first search processing unit 11, the second search processing unit 12, the sound output unit 13, and the input/output unit 14.

Alternatively, each of the first search processing unit 11, the second search processing unit 12, the sound output unit 13, and the input/output unit 14 may be implemented by dedicated hardware. Some or all of the components of each unit may be implemented by general-purpose or dedicated circuitry, a processor, or a combination thereof. These components may be formed by a single computer chip or by a plurality of computer chips connected to each other through a bus. Some or all of the components of each unit may be implemented by a combination of the above-mentioned circuitry or the like and a program(s). Further, for the processor, a CPU (Central Processing Unit), a GPU (Graphics Processing Unit), an FPGA (Field-Programmable Gate Array), a quantum processor (quantum computer control chip), or the like may be used.

Further, in the case where some or all of the components of the measuring apparatus 10 are implemented by a plurality of information processing apparatuses, circuits, or the like, the plurality of information processing apparatuses, circuits, or the like may be disposed at one place in a concentrated manner or disposed over a plurality of places in a distributed manner. For example, the information processing apparatuses, circuits, or the like may be implemented as a client-server system, a cloud computing system, or the like, in which the components or the like are connected to each other through a communication network. Further, the functions of the measuring apparatus 10 may be provided in the form of SaaS (Software as a Service).

An overview of processes performed by the measuring apparatus 10 will be described hereinafter with reference to FIG. 2. FIG. 2 shows an example of a display window output to the input/output unit 14. The input/output unit 14 displays a choice display area for receiving an input from the user on its screen. For example, as shown in FIG. 2, this choice display area may be buttons showing a text message “I could hear the sound” or a text message “I couldn't hear the sound”. The choice display area is not limited to the above-described example, and may be any area or the like by which it is possible to receive a response from the user to the output measurement sound. For example, the choice display area may be composed of, for example, an “OK” button and an “NG” button.

Further, as shown in FIG. 2, the input/output unit 14 displays various messages related to the measurement on its screen. By visually checking the screen of the input/output unit 14, the user can proceed with the measurement process according to the process performed by the measuring apparatus 10.

The measuring apparatus 10 outputs a measurement sound according to a predetermined binary tree. As will be described later, a volume level indicating the volume of a measurement sound is associated with each node of the binary tree. When the user heard a sound output from the sound output unit 13, he/she presses the “I could hear the sound” button, and when the user did not hear the sound, he/she presses the “I couldn't hear the sound” button. In this way, the user responds to the measuring apparatus 10 with information indicating that he/she could hear the measurement sound or could not hear the measurement sound.

Note that the input/output unit 14 may display only the “I could hear the sound” button, and when the user did not hear the sound, the input/output unit 14 may receive no input from the user. In this case, the measuring apparatus 10 determines that the user could not hear the sound when a predetermined time has elapsed without the “I could hear the sound” button being pressed after the measurement sound is output.

Upon receiving the response of the user, the measuring apparatus 10 outputs the next measurement sound from the sound output unit 13 according to the binary tree. In response to this next measurement sound, the user responds with information indicating whether or not the user heard the measurement sound in substantially the same manner as the above-described response. The measuring apparatus 10 repeatedly performs the above-described processes up to the convergence point at which the binary tree converges. When the measuring apparatus 10 reaches the convergence point, it determines the hearing ability of the user based on the volume level at the convergence point.

By performing the above-described processes, the measuring apparatus 10 measures the hearing ability of the user in a predetermined measurement range. The predetermined measurement range may be unchanged or changed as appropriate. The measurement range can correspond to, for example, a range of volumes with which the measuring apparatus 10 can output a sound from the sound output unit 13 (earphone). The measurement range can be set by using the maximum volume and the minimum volume among the volumes with which the measuring apparatus 10 can output a sound from the sound output unit 13. Note that the measurement range may be set to a range different from the range of volumes with which the measuring apparatus 10 can output a sound from the sound output unit 13. For example, the measurement range may be set to a range smaller than the range of volumes with which the measuring apparatus 10 can output a sound from the sound output unit 13.

Each of the components and structures of the measuring apparatus 10 will be described in detail by referring to FIG. 1 again. Unlike the related art described above with reference to FIG. 6, the measurement range of the hearing ability is divided into a plurality of groups in this embodiment. The first search processing unit 11 performs a first search process in which one of the plurality of groups is selected as a selected group in response to a response of the user. The selected group selected by the first search processing unit 11 is used in a second search process.

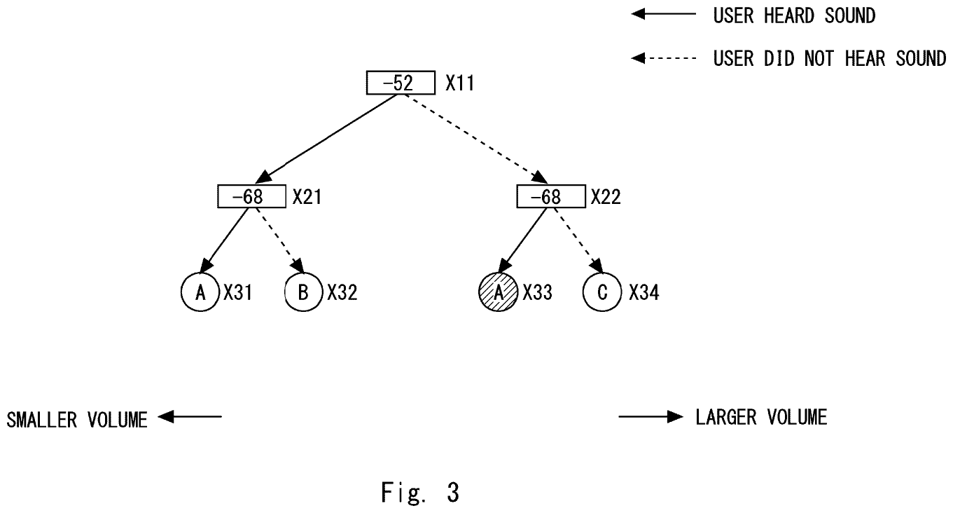

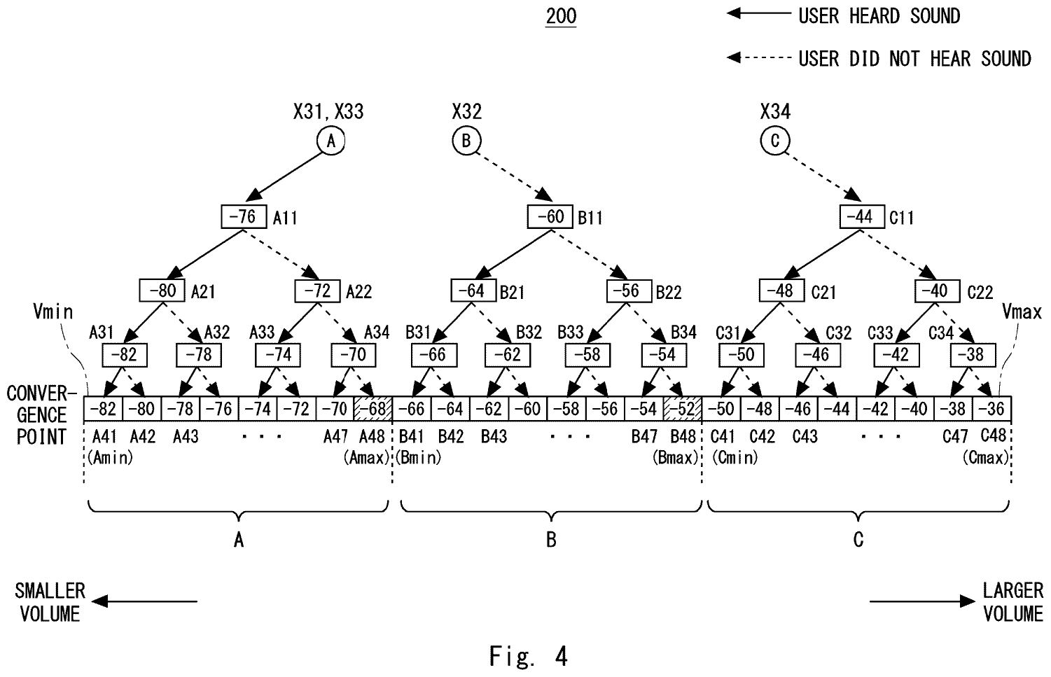

A first binary tree 100 used in the first search process will be described with reference to FIG. 3. FIG. 3 shows the first binary tree 100. Further, the following description will be given by referring to FIG. 4 as appropriate. FIG. 4 shows a second binary tree 200 used in the second search process. The first and second binary trees 100 and 200 may be stored in advance in a storage device or the like (not shown).

As shown in FIG. 3, the first binary tree 100 has nodes X11, X21, X22, . . . , and X34. The volume level of a measurement sound output by the measuring apparatus 10 is associated with each node. For example, the volume level of the highest-level node X11 is −52 dB.

The first binary tree 100 is configured so that the volume levels of measurement sounds become smaller from higher-level nodes to lower-level nodes. Therefore, the first search processing unit 11 outputs measurement sounds in such a manner that the deeper the position in the first binary tree 100 is, the smaller the volume level of the measurement sound becomes. In this way, the user can perform hearing-ability measurement without having any feeling of wrongness.

The first binary tree 100 has a number of nodes corresponding to the number of dividing points at which the measurement range of the hearing ability is divided. Note that the measurement range may indicate a range from the maximum level of the hearing ability, which is what the measuring apparatus 10 is to measure, to the minimum level thereof. Note that the measurement range corresponds to a range in which there are convergence points at which the second binary tree 200 converges in FIG. 4. In the second binary tree 200, the measurement range is set by using a maximum volume Vmax and a minimum volume Vmin. A node C48 corresponds to the maximum volume Vmax, and its volume level is −36 dB. Further, a node A41 corresponds to the minimum volume Vmin, and its volume level is −82 dB. Therefore, the measurement range is from −82 dB to −36 dB.

Further, as shown in FIG. 4, the measurement range shown by the second binary tree 200 is divided into three groups. The number of dividing points at which the measurement range is divided is two. As shown in FIG. 3, the first binary tree 100 has three nodes X11, X21 and X22 according to the number of dividing points. For example, when the number of dividing points is three or more, the first binary tree 100 may further have nodes lower than the nodes X21 and X22, i.e., nodes branched from the nodes X21 and X22. That is, the larger the number of dividing points is, the deeper the first binary tree 100 may become. The plurality of groups correspond to the binary trees A to C shown in FIG. 4. The binary trees A to C will be described later in detail.

Further, a volume level that serves as a boundary value in the second binary tree 200 is associated with each of the plurality of nodes of the first binary tree 100. Note that the boundary value indicates a volume level associated with a boundary node located at a boundary between groups of the second binary tree 200. In FIG. 4, shaded nodes B48 and A48 are used as boundary nodes.

The volume levels of the nodes B48 and A48, which are boundary nodes, are referred to as a first volume level and a second volume level, respectively. The first volume level is −52 dB corresponding to the node B48, and the second volume level is −68 dB corresponding to the node A48.

The first binary tree 100 has nodes corresponding to the first and second volume levels. Specifically, in the first binary tree 100, a volume level of −52 dB, which is the first volume level, is associated with the node X11. Further, a volume level of −68 dB, which is the second volume level, is associated with each of the nodes X21 and X22. Regardless of whether the response of the user at the node X11 is “I could hear the sound” or “I couldn't hear the sound”, the first search processing unit 11 outputs a measurement sound of the same volume level at the node X21 or X22. Note that, for example, when the number of dividing points is three, a measurement sound of a third boundary value is associated with nodes lower than the nodes X21 and X22, i.e., nodes branched from the nodes X21 and X22.

The first search process will be described in a concrete manner by using the first binary tree 100. Firstly, the first search processing unit 11 outputs a measurement sound having the first volume level (−52 dB) at the highest-level node X11. When there is a response from the user, indicating that he/she heard the sound, the first search processing unit 11 proceeds to the node X21 and outputs a measurement sound having the second volume level (−68 dB). The first search processing unit 11 receives a response of the user to this measurement sound.

The first search processing unit 11 determines whether or not there is an inconsistency between the response to the measurement sound having the first volume level and the response to the measurement sound having the second volume level. When the first search processing unit 11 determines that there is an inconsistency, it determines that an error has occurred and performs an error process. When the first search processing unit 11 determines that there is no inconsistency, it continues the search according to the first binary tree 100.

At the node X21, no inconsistency occurs in the content of the response either when there is a response from the user, indicating that he/she heard the sound or when there is a response indicating that he/she did not hear the sound. Therefore, when there is a response from the user, indicating that he/she heard the sound, the first search processing unit 11 proceeds to the node X31 and selects the binary tree A as the selected group. Further, when there is a response from the user, indicating that he/she did not hear the sound, the first search processing unit 11 proceeds to the node X32 and selects the binary tree B as the selected group.

On the other hand, assume that when a measurement sound having the first volume level is output at the highest-level node X11, there is a response from the user, indicating that he/she did not hear the sound. The first search processing unit 11 proceeds to the node X22 and outputs a measurement sound having the second volume level. The first search processing unit 11 receives a response to the measurement sound having the second volume level.

Here, assume that there is a response from the user, indicating that he/she heard the sound. In this case, although the user responded with information indicating that he/she did not hear the sound having the first volume level, the user has responded with information indicating that he/she heard the sound having the second volume level smaller than the first volume level. In this case, there is a possibility that the user made an incorrect response at the node X11 or made an incorrect response at the node X22. Alternatively, there is a possibility that the user made an incorrect response at both the nodes X11 and X22.

Therefore, the first search processing unit 11 determines that there is an inconsistency between the response to the measurement sound having the first volume level and the response to the measurement sound having the second volume level. The first search processing unit 11 proceeds to the next node X33 and performs an error process. In FIG. 3, the node X33 is indicated by shading.

As the error process, for example, the first search processing unit 11 assumes that the measurement result at the node X22, which was output later than the other, is correct, and selects the binary tree A as the selected group as shown in FIG. 3. Alternatively, the first search processing unit 11 may perform the first search process again as the error process. In this case, the first search processing unit 11 returns to the node X11, outputs a measurement sound having the first volume level again, and receives a response to this measurement sound again. In this way, the user may make a correct response in the measurement which has been performed again.

Note that when there is a response from the user, indicating that the user did not hear the sound having the second volume level at the node X22, the first search processing unit 11 determines that there is no inconsistency in this response. The first search processing unit 11 proceeds to the node X34 and selects the binary tree C as the selected group.

Note that although an example in which the number of dividing points at which the measurement range is divided is two has been used in this embodiment, the number of dividing points is not limited to two. The number of dividing points may be three or more. Therefore, the measurement range may be divided into four or more groups. The first binary tree 100 may be configured so that its depth increases as the number of dividing points increases. In this case, the first search processing unit 11 may perform an error process by making a decision by using a predetermined determination condition(s). For example, the first search processing unit 11 determines, based on responses to three or more measurement sounds having different volume levels, whether or not there is an inconsistency in the responses of the user. The first search processing unit 11 performs an error process depending on the determination result. The first search processing unit 11 selects one group from the second binary tree 200 by, for example, performing the first search process again in the error process. The first search processing unit 11 may use various determination conditions to perform the error process.

Further, although the first binary tree 100 is configured so that the volume levels of measurement sounds become smaller from higher-level nodes to lower-level nodes in this embodiment, the configuration of the first binary tree 100 is not limited to this example. For example, a volume level at a higher-level node may be lower than that at a lower-level node. Further, when a binary tree having a depth deeper than that of the first binary tree 100 according to this embodiment is used, there may be a lower-level node associated with a volume level equal to that of a higher-level node.

The description will be continued by referring to FIG. 1 again. The second search processing unit 12 performs a second search process in which it repeats a binary tree search in the selected group by using a second binary tree 200 until it converges, and thereby determines the hearing ability of the user. The selected group is one of the binary trees A to C selected in the first search process. Similarly to the first binary tree 100, the second binary tree 200 may be stored in advance in a storage device (not shown).

The second binary tree 200 will be further described with reference to FIG. 4. As described above, the second binary tree 200 corresponds to a measurement range which is defined by using a maximum volume Vmax and a minimum volume Vmin. Further, the second binary tree 200 includes binary trees A to C which divide the measurement range into three parts. The binary tree A has nodes A11 to A48; the binary tree B has nodes B11 to B48; and the binary tree C has nodes C11 to C48. The second search processing unit 12 performs a binary tree search from the highest-level node until it reaches a convergence point by using the binary tree which has been selected from among the binary trees A to C in the first search process.

The convergence points are indicated by nodes A41, A42, . . . , and C48. Like the other nodes, a volume level of a measurement sound is associated with each convergence point. The second search processing unit 12 determines the hearing ability of the user based on the volume level associated with the convergence point. Note that although convergence points are defined at intervals of 2 dB in FIG. 4, the intervals between convergence points are not limited to 2 dB. The intervals between convergence points may be smaller than 2 dB or larger than 2 dB. Further, the intervals between convergence points may not be defined as constant intervals.

Further, each of the binary trees A to C has a volume range defined by an upper limit volume and a lower limit volume, and these volume ranges are defined so as not to overlap each other. For example, the binary tree A has a volume range defined by an upper limit volume Amax and a lower limit volume Amin. The upper limit volume Amax is −68 dB associated with the node A48. Further, the lower limit volume Amin is −82 dB associated with the node A41. Therefore, the binary tree A has a volume range from −82 dB to −68 dB. As shown as the nodes A41 to A48, the binary tree A has eight convergence points at intervals of 2 dB.

Each of the binary trees B and C has a configuration substantially the same as that of the binary tree A. The binary tree B has nodes B48 and B41 corresponding to an upper limit volume Bmax and a lower limit volume Bmin, respectively. The upper and lower limit volumes Bmax and Bmin are −52 dB and −66 dB, respectively. Therefore, the binary tree B has a volume range from −66 dB to −52 dB. Similarly, the binary tree C has nodes C48 and C41 corresponding to an upper limit volume Cmax and a lower limit volume Cmin, respectively. The upper and lower limit volumes Cmax and Cmin are −36 dB and −50 dB, respectively. Therefore, the binary tree C has a volume range from −50 dB to −36 dB.

As described above, the binary trees A to C are defined so that their volume ranges do not overlap each other. By defining a volume range so as not to overlap those of other groups, the second search processing unit 12 can reduce the number of responses that the user needs to make for the second search process. Therefore, the second search processing unit 12 can efficiently perform the second search process. Further, by reducing the number of responses, the burden on the user can be reduced. Note that the definition of a plurality of groups is not limited to the above-described example. That is, a plurality of groups may be defined so that their volume ranges overlap each other.

The description will be continued by referring to FIG. 1 again. The sound output unit 13 is a sound output device that outputs a measurement sound. The sound output unit 13 may be, for example, a listening device such as an earphone(s) or a headphone. The sound output unit 13 may be configured so as to be able to communicate with the measuring apparatus 10 through a wire or wireless. The sound output unit 13 may be, for example, a wireless earphone(s) which is connected to the measuring apparatus 10 in conformity with wireless communication standards such as Bluetooth (Registered Trademark). Further, the sound output unit 13 may be left-right separated type (completely wireless type) earphones of which left and right output units are physically independent of each other, or left-right integrated type earphones, such as neckband type earphones, of which left and right output units are physically connected to each other.

Note that they are merely examples, and other types of sound output devices may be provided as the sound output unit 13. Further, the sound output unit 13 may output a sound (or a voice) other than the measurement sounds. For example, the sound output unit 13 may be configured to output a sound (or a voice) related to the measurement process.

The input/output unit 14 is an input/output device that receives an input of a response to a measurement sound from a user. The input/output unit 14 includes an input unit that receives an input from the user and an output unit that outputs display information for the user. In this embodiment, a display device equipped with a touch panel that enables a user to perform an input operation by touching the touch panel with his/her finger(s) or the like is used as an example of the input/output unit 14. The input/output unit 14 is not limited to this example. For example, an input unit and an output unit may be provided separately from each other as the input/output unit 14. For example, the input unit may be an input device such as a keyboard, and the output unit may be a display device such as a liquid-crystal display panel. The input/output unit 14 is not limited to these examples. That is, the input/output unit 14 may be configured in various forms. For example, the input/output unit 14 may be configured so that the content of a response of a user can be input by using user's voice. The input unit may be formed by using a physical button or a touch sensor disposed on the earphone. Further, the input unit may be configured so that a user or the like can input information or the like by using an existing physical button already provided in the display device equipped with a touch panel according to this embodiment.

The input/output unit 14 outputs information related to the measurement process and notifies the user thereof. For example, as shown in FIG. 2, the input/output unit 14 displays a message such as “When you hear a sound, please tap on ‘I could hear the sound”’. In this way, the input/output unit 14 urges the user to respond to the measurement sound.

Further, after the measurement sound is output, the input/output unit 14 receives a response from the user to the measurement sound. The input/output unit 14 receives a response from the user in response to, for example, the pressing of the “I could hear the sound” button or the “I couldn't hear the sound” button shown in FIG. 2. Alternatively, the input/output unit 14 may presume that the user could not hear the measurement sound when a predetermined time (e.g., five seconds) or longer has elapsed after the measurement sound is output. When the input/output unit 14 determines that there is no response, it may output the same measurement sound again.

### (Processing of Measurement Apparatus 10)

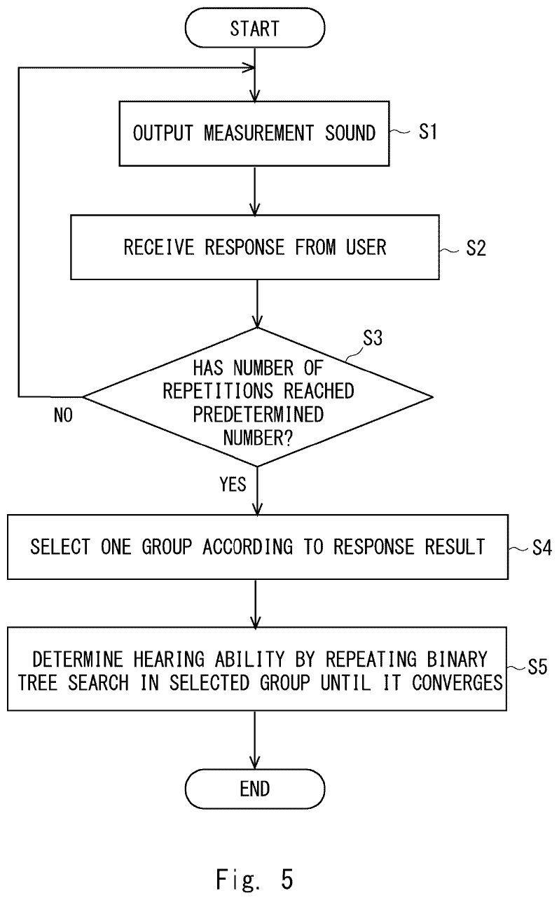

Next, a measurement process performed by the measuring apparatus 10 according to this embodiment will be described with reference to FIG. 5. FIG. 5 is a flowchart showing the measurement process performed by the measuring apparatus 10.

The measuring apparatus 10 performs the measurement process by using the first binary tree 100 and the second binary tree 200 shown in FIGS. 3 and 4, respectively. Specifically, the first search processing unit 11 performs the first search process by using the first binary tree 100 (S1 to S4). The second search processing unit 12 performs the second search process by using the second binary tree 200 (S5).

Firstly, the first search processing unit 11 makes, i.e., instructs, the sound output unit 13 output a measurement sound (S1). The volume level of the measurement sound is determined according to the first binary tree 100. For example, at the start of the measurement, the first search processing unit 11 outputs a measurement sound having a volume level associated with the highest-level node X11 of the first binary tree 100.

The user inputs a response indicating whether or not he/she heard the measurement sound to the measuring apparatus 10 through the input/output unit 14. For example, when the user heard the measurement sound, he/she presses (taps on) the “I could hear the sound” button on the window displayed on the input/output unit 14. Further, when the user has not heard the measurement sound, he/she presses the “I couldn't hear the sound” button on the display window. The first search processing unit 11 receives the response from the user (S2).

The first search processing unit 11 determines whether or not the number of executions of the processes in the steps S1 and S2 has reached a predetermined number of times (S3). The predetermined number of times is defined according to the number of division points at which the measurement range of the hearing ability is divided. In this example, since the number of division points is two, the predetermined number of times is two. When there are more division points, the predetermined number of times may be set to a larger number. When the number of executions of the processes in the steps S1 and S2 has not yet reached the predetermined number of times (No in S3), the process returns to the step S1. The first search processing unit 11 repeats the processes in the steps S1 and S2 until the number of executions of the processes reaches the predetermined number of times.

For example, the first search processing unit 11 outputs a measurement sound having a volume of −52 dB corresponding to the node X11 of the first binary tree 100, and then receives a response thereto from the user. The first search processing unit 11 proceeds to the node X21 or X22 according to the content of the response. The first search processing unit 11 outputs a measurement sound having a volume of −68 dB corresponding to the node X21 or X22, and receives a response thereto from the user. In this way, the number of executions of the processes in the steps S1 and S2 reaches the predetermined number of times.

When the number of executions of the processes reaches the predetermined number of times (Yes in S3), the first search processing unit 11 selects one group according to the result of the response of the user (S4). The first search processing unit 11 determines whether or not there is an inconsistency in the content of the response, and selects a group according to the determination result. When the first search processing unit 11 determines that there is an inconsistency, it determines that an error has occurred and performs an error process. On the other hand, when the first search processing unit 11 determines that there is no inconsistency, it selects a binary tree according to the first binary tree 100.

For example, assume that the user responds with information indicating that he/she heard the measurement sound at the node X22. In this case, there is an inconsistency between the response at the node X11 and the response at the node X22. In this case, the first search processing unit 11 proceeds to the node X33 and performs an error process. As the error process, the first search processing unit 11 assumes that the measurement result at the node X22, which was output later than the other, is correct, and selects the binary tree A. Alternatively, the first search processing unit 11 may perform the first search process again as the error process. In this case, the first search processing unit 11 returns to the node X11, outputs a measurement sound having the volume level of −52 dB again, and receives a response to this measurement sound again. In this way, the first search processing unit 11 selects one binary tree from among the binary trees A to C.

Next, the second search processing unit 12 determines the hearing ability of the user by repeating the binary tree search in the selected group until it converges (S5). Specifically, the second search processing unit 12 performs the binary tree search by using the second binary tree 200. The second search processing unit 12 performs the search, among the binary trees A to C, in the selected binary tree until it converges. The second search processing unit 12 determines the hearing ability of the user based on the volume level corresponding to the node at the convergence point. In this way, the measurement of the hearing ability of the user is completed. The measuring apparatus 10 may output the measurement result by using the sound output unit 13 or the input/output unit 14. Further, the measuring apparatus 10 may start the measurement process again while changing the frequency of the measurement sound. Further, the measuring apparatus 10 may start a measurement process for the ear opposite to the ear for which the measurement has been completed.

As described above, the measuring apparatus 10 according to this embodiment measures the hearing ability of the user by performing binary tree searches using the first binary tree 100 and the second binary tree 200. In the second binary tree 200, the measurement range of the hearing ability is divided into a plurality of groups, and the first search processing unit 11 selects one group from among the plurality of groups as a selected group. Further, the second search processing unit 12 determines the hearing ability of the user by repeating the binary tree search in the selected group until it converges.

By the above-described configuration, the measuring apparatus 10 according to this embodiment can prevent the result of measurement from being significantly deviated from a correct one, which would otherwise be caused by a mistake in which the user has incorrectly recognized other sounds as the measurement sound or a mistake in regard to the pressing of the button.

### (Example of Hardware Configuration)

Each of the functional components of the above-described measuring apparatus 10 can be implemented by hardware that implements the functional component (e.g., a hardwired electronic circuit or the like) or by a combination of hardware and software (e.g., a combination of an electronic circuit and a program for controlling it or the like). For example, the present disclosure may also be implemented by causing a CPU (Central Processing Unit) to execute a computer program.

In the above-described examples, the program includes a set of instructions (or software codes) that, when read into a computer, causes the computer to perform one or more of the functions described in the example embodiments. The program may be stored in a non-transitory computer readable medium or in a tangible storage medium. By way of example rather than limitation, a computer readable medium or a physical storage medium may include a random-access memory (RAM), a read-only memory (ROM), a flash memory, a solid-state drive (SSD), or other memory technology, a CD-ROM, a digital versatile disk (DVD), a Blu-ray (Registered Trademark) disc or other optical disc storages, a magnetic cassette, magnetic tape, and a magnetic disc storage or other magnetic storage devices. The program may be transmitted on various types of transitory computer readable media or communication media. By way of example rather than limitation, transitory computer readable media or communication media may include electrical, optical, acoustic, or other forms of propagation signals.

Note that the present disclosure is not limited to the above embodiments, and they may be modified as appropriate without departing from the scope and spirit of the disclosure.

The present disclosure contributes to the realization of “Good Health and Well-Being” of Sustainable Development Goals (SDGs) and includes matters that contribute to the creation of value through healthcare products and services.

The measuring apparatus, the measurement method, and the program according to the embodiment can prevent a result of measurement of a hearing ability from being significantly deviated from a correct.

The present disclosure can be applied to a measuring apparatus or the like used for the measurement of a hearing ability.

What is claimed is:

1. A measuring apparatus configured to measure a hearing ability of a user by performing a binary tree search based on responses of the user to measurement sounds, comprising:
a first search processing unit configured to select, as a selected group, one of a plurality of groups obtained by dividing a measurement range of the hearing ability into the plurality of groups; and
a second search processing unit configured to repeat the binary tree search in the selected group until the binary tree search converges, and thereby determine the hearing ability.

2. The measuring apparatus according to claim 1, wherein the first search processing unit selects the selected group by performing a search in a binary tree having a number of nodes corresponding to the number of dividing points at which the measurement range is divided.

3. The measuring apparatus according to claim 1, wherein the first search processing unit selects the selected group by performing a search in a binary tree configured so that volume levels of measurement sounds become smaller from a higher-level node to a lower-level node.

4. The measuring apparatus according to claim 1, wherein each of the plurality of groups has a volume range defined by a higher limit volume and a lower limit volume, and the volume ranges are defined so as not to overlap each other.

5. The measuring apparatus according to claim 3, wherein the first search processing unit determines whether or not there is an inconsistency between a response to a measurement sound at the higher-level node and a response to a measurement sound at the lower-level node, and when the first search processing unit determines that there is an inconsistency, the first search processing unit determines that an error has occurred and performs an error process.

6. The measuring apparatus according to claim 5, wherein as the error process, the first search processing unit assumes that a measurement result at the lower-level node is correct, and selects the selected group having a smaller volume level branched from the lower-level node.

7. A measurement method for measuring a hearing ability of a user by performing a binary tree search based on responses of the user to measurement sounds, comprising:
a first search processing step of selecting, as a selected group, one of a plurality of groups obtained by dividing a measurement range of the hearing ability into the plurality of groups; and
a second search processing step of repeating the binary tree search in the selected group until the binary tree search converges, and thereby determining the hearing ability.

8. A non-transitory computer readable medium storing a program for causing a computer of a measuring apparatus configured to measure a hearing ability of a user by performing a binary tree search based on responses of the user to measurement sounds to perform a measurement method,
the measurement method comprising:
a first search processing step of selecting, as a selected group, one of a plurality of groups obtained by dividing a measurement range of the hearing ability into the plurality of groups; and
a second search processing step of repeating the binary tree search in the selected group until the binary tree search converges, and thereby determining the hearing ability.
